(19) United States
(12) Patent Application Publication
(10) Pub. No.: US 2011/0152820 A1
(43) Pub. Date: Jun. 23, 2011

(54) BARRIER COATINGS FOR FLUIDS CONTACTING MEDICAL DEVICES

(75) Inventors: Sarnath Chattaraj, Simi Valley, CA (US); Kiem Dang, Thousand Oaks, CA (US); Poonam S. Gulati, La Canada, CA (US)

(73) Assignee: MEDTRONIC MINIMED, INC., Northridge, CA (US)

(21) Appl. No.: 12/974,914

(22) Filed: Dec. 21, 2010

Related U.S. Application Data
(60) Provisional application No. 61/289,226, filed on Dec. 22, 2009.

(51) Int. Cl. A61L 31/14; B65D 81/24; A61J 1/14; B05D 3/06; B65B 1/04

(52) U.S. Cl. 604/403; 206/205; 427/569; 427/248.1; 141/1

(57) ABSTRACT

The invention relates to methods and materials that, for example, function to increase the barrier properties of containers including polymeric drug medication reservoirs and related containers such as infusion set tubing. Embodiments of the invention include aqueous container systems having containers coated with a composition selected to have one or more material properties including an ability to reduce the diffusion or permeation of compounds such as oxygen, carbon dioxide, and preservatives (e.g. phenol, benzyl alcohol and m-cresol) into or through a wall of the container.

### CROSS REFERENCE TO RELATED APPLICATIONS

This application claims priority under Section 119(e) from U.S. Provisional Application Ser. No. 61/289,226 filed Dec. 22, 2009, the contents of which are incorporated herein by reference.

### BACKGROUND OF THE INVENTION

1. Field of the Invention

This invention relates to container systems used to store and/or deliver aqueous medications.

2. Description of Related Art

Therapeutic polypeptides such as insulin and human growth hormone are commonly stored in formulations disposed within small containers or reservoirs. Such formulations typically include one or more preservatives that function to extend the shelf-life of the therapeutic agent. Because terminal sterilization is not appropriate for many therapeutic formulations containing proteins, peptides and the like, preservatives are essential ingredients in these formulations, particularly in those formulations that are administered parenterally.

Insulin is a well known parenterally administered therapeutic polypeptide that is used, for example, by diabetics to regulate their blood glucose levels. Containers of insulin and the like are normally refrigerated at a temperature of about 5° C. when stored in hospitals or pharmacies. However, they are also often maintained at room temperature for periods of up to one month during their therapeutic administration (e.g. insulin formulations disposed within an ambulatory infusion pump worn by a diabetic patient). In such situations, it is important that the concentration of preservatives in these formulations be maintained over the storage period. Specifically, if the concentration of a preservative drops below a certain level, the therapeutic agent may not be sufficiently preserved over its period of use.

Traditional reservoirs for storing polypeptides such as insulin comprise glass or plastic/polymeric materials. Glass materials are popular due to their high barrier properties. Plastic materials are popular because of their low cost, low density and the ease with which they can be processed into large range of different products. Unfortunately however, both reservoir materials exhibit limitations. Glass reservoirs can break easily, for example when being manipulated by non-medical personnel such as individuals suffering from juvenile (Type I) diabetes. In addition, while polymeric materials can function to provide a barrier to oxygen and certain other gases, these materials do not always provide sufficient barrier against preservatives and water, agents which can be absorbed into and/or diffuse through polymeric materials.

As noted above, while exhibiting a number of desirable characteristics including low cost and durability, polymeric reservoir materials suffer from a number of disadvantages as compared to glass reservoir materials including suboptimal gas barrier properties. For these reasons, there is a need for methods and materials that can facilitate the storage of therapeutic polypeptides within plastic/polymeric reservoirs in the wide variety of situations in which these agents are used.

### SUMMARY OF THE INVENTION

Embodiments of the invention provide methods and materials that function to increase the barrier properties of containers including polymeric drug medication reservoirs and related containers such as infusion set tubing. As disclosed herein, by coating these types of containers with a composition selected to have one or more material properties including an ability to reduce the diffusion of compounds such as oxygen, carbon dioxide, and preservatives (e.g. phenol, benzyl alcohol and m-cresol) into or through a wall of the container, the activity of a therapeutic agent stored within the container can be preserved.

An illustrative embodiment of the invention is an aqueous container system comprising a container formed from a polymeric material, an aqueous solution disposed within the container and comprising a polypeptide combined with a compound selected for its ability to preserve a biological activity of the polypeptide. In this embodiment of the invention, at least one surface of the container is coated with a layer of material selected for its ability to inhibit diffusion of the compound and gases into or through the container. A variety of polymeric compositions can be used to form the container, for example a polyethylene, a polypropylene, a polyester, a cyclic olefin, a polyurethane, a poly(vinylchloride), a polycarbonate, a polystyrene, a polyacrylate, or a co-polymer material composed of aliphatic cyclic or bicylic hydrocarbons with 5 to 7 membered ring or rings, and ethylene or propylene, polyamide. In addition, a variety of compositions can be used to form the coating layer. In illustrative embodiments of the invention, the coating layer comprises at least one barrier layer of materials selected from a polyp-xylylene) compound, a silicon compound, a fluoropolymer compound, a ethylene vinyl alcohol compound, or its copolymers, ethylene vinyl alcohol, a polyvinylidine compound, a tetrahedral amorphous carbon compound, or a metal compound such as a metal oxide, a metal carbide, a metal oxynitrile, a metal oxyboride or combinations thereof.

A variety of containers commonly used to hold aqueous medications are encompassed by embodiments of the invention. In typical embodiments of the invention, the container comprises a medication storage vial, a medication reservoir disposed within a medical device, or infusion set tubing. In this context, the coating layer or layers can be disposed, for example, on the outside of the container, on the inside of the container that contacts the fluid medication, or between a first layer and a second layer of polymeric material that forms the container. In certain embodiments of the invention, a polymeric container material and/or its coating layer comprise a biodegradable compound. Optionally, the polymeric material and the coating layer are transparent.

The biological activity of a variety of therapeutic polypeptides or polynucleotides can be protected by embodiments of the invention. Illustrative polypeptides comprise insulins, interferons, and human growth hormones. In addition, the function of a variety of preservative compounds used in aqueous medications are enhanced by embodiments of the invention. In typical embodiments of the invention, the compound comprises an antimicrobial agent. In specific illustrative embodiments of the invention, the compound comprises a phenol, a benzyl alcohol or a cresol.

Another embodiment of the invention is a method of inhibiting the loss of a biological activity of a polypeptide stored within a container by storing the polypeptide within an aqueous container system disclosed herein. An illustrative aqueous container system can comprise, for example, a container formed from a polymeric material, and an aqueous solution disposed within the container which includes a polypeptide combined with a compound selected for its ability to preserve a biological activity of the polypeptide. In such embodiments of the invention, a surface of the container is coated with a layer of material selected for its ability to inhibit diffusion of the compound and/or gases into or through a container matrix (e.g. a reservoir wall) so that the preservative activity and/or concentration of the compound is maintained in a manner that inhibits the loss of a biological activity of the polypeptide under a variety of conditions and/or inhibits microbial growth in the solution.

Certain embodiments of the invention are designed for use with polypeptides, that, at times during their use, are stored at temperatures above 5° C. or 10° C. (e.g. room temperature) for at least 24, 48, 72 or more hours prior to their administration to a patient. For example, in certain embodiments of the invention, the polypeptide comprises insulin and the container is a medication reservoir disposed within a medication infusion pump having dimensions smaller than 15×15 centimeters and designed to be worn by a user during daily activities (and/or infusion set tubing that is operatively coupled to such a reservoir). In certain embodiments of the invention, the medication infusion pump is worn externally and suited for ambulatory use. In such embodiments, the pump can be operably coupled to an interface that facilitates its attachment to the user, wherein the interface comprises a clip, a strap, a snap, a clamp or an adhesive strip. Alternatively the medication infusion pump can be one designed for implantation within a diabetic individual.

Yet another embodiment of the invention is a method of making an aqueous container system having the disclosed barrier properties. One such embodiment comprises, for example, the steps of forming a container from a polymeric material, coating a surface of the container with a layer or layers of material(s) selected for its ability to function as a barrier that inhibits diffusion of compounds and gases into or through the container such that compounds can be maintained at a concentration sufficient to inhibit loss of a biological activity of a polypeptide disposed within the container. A solution is then placed within the container, with illustrative solutions comprising water and a polypeptide combined with a compound selected for its ability to preserve a biological activity of the polypeptide and/or inhibit microbial growth in the solution. In this way, a variety of aqueous container systems having the disclosed barrier properties can be made.

In illustrative methods for making an aqueous container system, the surface of the container can be modified by applying a thin film of material. Such methods can include, for example, a process comprising plasma surface treatment. Optionally, a surface of the container is modified by a process comprising chemical and/or physical vapor deposition. In certain embodiments of the invention, the surface of the container is coated with the layer of material under a vacuum, for example at a pressure at least 10% below atmospheric pressure.

Other objects, features and advantages of embodiments of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE FIGURES

FIG. 1 provides a diagram showing cross sections of embodiments of the invention where a barrier material is coated at different locations on a circular container (e.g. a medication reservoir or infusion set tubing). The center circle in these figures represents the space in which a medication is stored. From left to right, the three diagrams show a coating disposed on the outside of the container, a single or multiple layer coating disposed on the inside of the container that contacts the fluid medication, or a single or multiple layer coating disposed between a first layer and a second layer of a polymeric material that forms the container.

FIG. 2 provides a diagram showing an infusion pump system including a medication reservoir and infusion set tubing.

### DETAILED DESCRIPTION OF THE EMBODIMENTS

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. A number of terms are defined below.

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

Before the present device systems and methods etc. are described, it is to be understood that this invention is not limited to the particular structure, methodology, protocol, composition etc., described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise. Thus, for example, reference to “a preservative” includes a plurality of such preservatives and equivalents thereof known to those skilled in the art, and so forth. All numbers recited in the specification and associated claims that refer to values that can be numerically characterized with a value other than a whole number are understood to be modified by the term “about”.

### ILLUSTRATIVE ASPECTS AND EMBODIMENTS OF THE INVENTION

As noted above, embodiments of the invention provide methods and materials designed to enhance the barrier properties of certain types of containers, for example drug medication reservoirs and infusion set tubing formed from polymeric/plastic materials. In particular, the biological activities of therapeutic agents such as insulin can be protected by coating these containers with one or more layers of a composition selected to have one or more material properties including an ability to reduce the permeation or diffusion of compounds such as oxygen, carbon dioxide, and preservatives (e.g. phenol, benzyl alcohol and m-cresol) into or through a wall of the container. A number of illustrative and non-limiting methods and materials of the invention are discussed below.

### Illustrative Aqueous Formulations of the Invention

Medications comprising proteins, peptides, and/or DNA molecules exhibit biological activities that are sensitive to a number of phenomena including microbial contamination as well as exposure to oxygen and carbon dioxide gases, compounds which can interact with and/or change the storage environment of the medication. Such interactions and/or changes to the medication environment can substantially shorten the shelf life of such medications. In addition, when kept in storage over a period of time during use, the concentrations of compounds in aqueous formulations (e.g. those comprising insulin and preservatives) should be kept at a relatively constant level. If, for example, the concentration of preservative (e.g. an antimicrobial agent) drops below certain levels, the medication may not be sufficiently preserved (e.g. may be degraded and/or lose biological activity). Similarly, the loss of water molecules within an aqueous formulation should also be minimized during storage in order to, for example, keep the relative concentrations of compounds in aqueous formulations at relatively constant levels.

The biological activity of a variety of therapeutic polypeptides or polynucleotides stored in aqueous environments can be protected by embodiments of the invention disclosed herein. Embodiments of the invention include an aqueous solution comprising a therapeutic agent such as a polypeptide (e.g. a protein such as insulin, a small peptide such as dipeptide alanyl-glutamine and the like) and/or a polynucleotide (e.g. a single or double stranded oligonucleotide, a SiRNA and the like) combined with a compound selected for its ability to preserve a biological activity of the therapeutic agent. A variety of therapeutic polypeptides can be used in embodiments of the invention including, for example, a human growth hormone (hGH), an interferon (e.g. a α, β or γ interferon), interleukins (e.g. IL-2 and its analogs) a clotting factor such as Factor VIII, and/or an insulin. Specific polypeptides suitable for use in the practice in embodiments of the present invention further include, for example, insulin analogs (e.g. LysB28ProB29-human insulin and AspB28 human insulin), interferon analogs such as IFN-βₛₑᵣ₁₇), as described in EPO 185459B1 (incorporated herein by reference) and hGH analogues such as those described in U.S. Pat. No. 5,849,535 (incorporated herein by reference). While specific embodiments of the invention are directed to stabilization of insulin and its analogs, the utility of the invention extends generally to all protein and polypeptide pharmaceuticals.

Water of suitable quality for use in the aqueous formulations of the invention is typically prepared either by distillation or by reverse osmosis. In addition to water, other ingredients can be included in the polypeptide pharmaceutical formulation embodiments of the present invention. Such additional ingredients can include, for example, preservatives, wetting agents, emulsifiers, antioxidants, bulking agents, tonicity modifiers, chelating agents, metal ions, oleaginous vehicles, other proteins (e.g., human serum albumin or gelatin) and a zwitterion (e.g., an amino acid such as betaine, taurine, arginine, glycine, lysine and histidine). Such additional ingredients, of course, should not adversely affect the overall stability of the pharmaceutical formulation embodiments of the present invention.

The United States Pharmacopeia (USP) is an official public standards-setting authority which states that preservatives such as anti-microbial agents in bacteriostatic or fungistatic concentrations must be added to a large number of aqueous preparations, for example those contained in multiple dose containers. Moreover, such preservatives must be present in adequate concentration at the time of use to prevent the multiplication of microorganisms inadvertently introduced into the preparation while withdrawing a portion of the contents with a hypodermic needle and syringe, or using other invasive means for delivery, such as pen injectors. Antimicrobial agents used in such formulations must be evaluated to ensure compatibility with all other components of the formula, and their activity should be evaluated in the total formula to ensure that a particular agent that is effective in one formulation is not ineffective in another.

A preservative is, in the common pharmaceutical sense, a substance that prevents or inhibits microbial growth and may be added to pharmaceutical formulation for this purpose to avoid consequent spoilage of the formulation by microorganisms. While the amount of the preservative contained within aqueous formulations is not great, it does effect the overall stability of the therapeutic agent. Consequently, the selection of components for use with an aqueous formulation systems can be problematic. As biologic-based drugs become an increasingly larger part of pharmaceutical product portfolios however, strategies for optimizing their formulations become increasingly important. In this context, there are a number of issues to take into consideration when selecting an antimicrobial agent. A key issue is understanding that there are potential incompatibilities with the protein itself, as well as potential incompatibilities with processing materials (tubing and filters), other formulation ingredients (polysorbate 80 and other polymers), and container materials. In this context, the function of a variety of preservative compounds used in aqueous medications are enhanced by embodiments of the invention.

M-cresol, phenol, and benzyl alcohol are the most widely used preservative agents used in protein products. They are generally used as single agents, although certain formulations such as insulin-protamine suspension products, can for example, contain both m-cresol and phenol. Cresols are organic compounds which are methylphenols. In organic chemistry, phenols, sometimes called phenolics, are a class of chemical compounds consisting of a hydroxyl group (—OH) bonded directly to an aromatic hydrocarbon group. The simplest of the class is phenol (C6H5OH). Phenol has been successfully used as a preservative in drug formulations for more than 50 years and is considered a safe and effective agent which complies with strict international requirements for preservatives in drug formulations. In its chemical structure, a cresol molecule has a methyl group substituted onto the benzene ring of a phenol molecule. There are three forms of cresols that are only slightly different in their chemical structure: ortho-cresol (o-cresol), meta-cresol (m-cresol), and para-cresol (p-cresol). These forms occur separately or as a mixture. The word tricresol can be used as a synonym for cresol where it means a mixture of o-, m- and p-cresols. Benzyl alcohol is an organic compound with the formula C6H5CH2OH. A detailed description of these and other preservatives suitable for use with embodiments of the invention is set forth in Remington's Pharmaceutical Sciences Lippincott Williams & Wilkins; Twenty-First edition (May 19, 2005) as well as Pharmaceutical Dosage Forms: Parenteral Medications, Vol. 1, 1992, Avis et al.

One or more stabilizers may be included in the aqueous formulations of the invention. If included, illustrative stabilizers useful in the practice of embodiments of the present invention include carbohydrates and/or polyhydric alcohols. Polyhydric alcohols include compounds such as sorbitol, mannitol, glycerol, and polyethylene glycols (PEGs). These compounds are straight-chain molecules. The carbohydrates, such as mannose, ribose, trehalose, maltose, inositol, and lactose, on the other hand, are cyclic molecules that may contain a keto or aldehyde group. These two classes of compounds have been demonstrated to be effective in stabilizing protein against denaturation caused by elevated temperature and by freeze-thaw or freeze-drying processes. Suitable carbohydrates include: galactose, arabinose, lactose or any other carbohydrate which does not have an adverse affect on a diabetic patient, i.e., the carbohydrate is not metabolized to form large concentrations of glucose in the blood. Such carbohydrates are well known in the art as suitable for use in formulations administered to diabetics.

It may also be desirable to add sodium chloride or other salts to adjust the tonicity of the pharmaceutical formulation, depending on the tonicifier selected. However, this is optional and depends on the particular formulation selected. Parenteral formulations must be isotonic or substantially isotonic otherwise significant irritation and pain would occur at the site of administration. In addition, in order to, for example, maintain the aqueous formulation at a particular pH range, a buffer may be used. The terms buffer, buffer solution and buffered solution, when used with reference to hydrogen-ion concentration or pH, refer to the ability of a system, particularly an aqueous solution, to resist a change of pH on adding acid or alkali, or on dilution with a solvent. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydronium or hydroxyl ion added does not exceed the capacity of the buffer system to neutralize it.

A wide variety of formulations can be made in accordance with embodiments of the invention. In one specific but non-limiting exemplary embodiment of an aqueous formulation, insulin is present at a concentration of about 250 to about 1000 U/ml, zinc is present at a concentration of about 0.07 μg/ml to about 0.09 μg/ml, m-cresol is present at a concentration of about 2.2 mg/ml, phenol is present at a concentration of about 0.9 mg/ml and glycerol is the isotonicity agent and is present at a concentration of about 16 mg/ml.

### Illustrative Container Materials and Structures of the Invention

Containers are an integral part of the formulation of a parenteral medication and may be considered a component, for there is no container that is totally insoluble or does not in some way affect the liquid it contains, particularly if the liquid is aqueous. Therefore, the selection of a container for a particular application (e.g. insulin used in an ambulatory infusion device) must be based on a consideration of the composition of the container, as well as of the solution, and the treatment to which it will be subjected.

Polypeptide medications, such as insulin or growth hormone, are commonly distributed in small containers or ampoules. Such ampoules normally comprise between 1.5 and 10 ml of ready-to-use medication. These ampoules are stored in stock, at the hospitals or pharmacies, and with the user. This means that the shelf-life must be sufficiently long to allow a full utilization of the therapeutic compound. As noted above, aqueous solutions or suspensions of medications, such as insulin or growth hormones, are normally provided with a preservative, such as phenol and/or benzyl alcohol and/or m-cresol. Addition of preservatives is necessary because a terminal sterilization of the container is not possible due to the sensitivity of medications containing proteins, peptides and/or DNA sequences. Medications in containers comprising more than one dose, e.g. for use in pen systems, are at a high risk of contamination. Therefore, preservatives are essential ingredients in such medications, in particular in medications for parenteral administration.

Plastic are popular containers for medications because of their low cost, low density and the ease with which they can be processed into a large range of different products. Plastic materials, however, suffer from a number of disadvantages in comparison to containers made from glass and metal materials. For example, a major disadvantage of such plastic materials is poor gas barrier properties. This is especially true for polypropylene and the polyethylene. In “Stability and sterility of biosynthetic human insulin stored in plastic insulin syringe for 28 days” by Tarr et al, American Can Society of Hospital Pharmacists, Vol. 48, pp 2631-2634 (1991) it is noted that tests on aqueous solutions stored in polypropylene-polyethylene syringes, exhibited losses of phenol, benzyl alcohol and m-cresol. From this 28 day study, it was determined that polypropylene-polyethylene syringes are not well suited for storing medications comprising phenol and/or benzyl alcohol and m-cresol due to the poor barrier properties of this material. Similarly, some containers made from terephthalic polyesters can provide a barrier against oxygen and other gases, but not against preservatives and water. In this context, an objective of embodiments of the present invention is to increase specific barrier properties of containers such as reservoirs for storing medications or infusion set tubing for delivery of medication from the device to the therapy site. The containers disclosed herein comprise barrier coatings that function to inhibit the diffusion of compounds such as gasses and preservatives such as phenol, benzyl alcohol and m-cresol through the containers, thereby providing provides a medication container suitable for long-time storage of aqueous medications.

A variety of polymeric materials can be used to make the containers of the invention. Illustrative suitable container materials comprise polyolefins (e.g. a cyclic olefin copolymer such as TOPAS), polyethylenes, polypropylenes, polycarbonates, polyamides, ethylenevinyl acetate copolymers, ethylene-methacrylate copolymers, polyvinyl chlorides, polystyrenes, polyesters, polyester amides, polyacrylic esters, polyvinylidene chlorides, polyurethanes, polyacetals, polycarbonates, silicones, polysulfones, polyimides, polycarbonates, polyacrylates, poly ether ether ketones (PEEK); or a co-polymer material composed of aliphatic cyclic or bicylic hydrocarbons with 5 to 7 membered ring or rings, and ethylene or propylene. These and other thermoplastics may be utilized either singularly or in combinations.

A specific illustrative embodiment of the invention is a medicament container for storing a liquid medicament comprising one or more active ingredients in water in combination with a preservative such as m-cresol and/or phenol and/or benzyl alcohol. The container comprises a distal and a proximal end portion and a wall, with at least two portions of the wall being coated with barrier polymer material. In this embodiment, the polymer wall portion comprises a polymer such as polypropylene, PET, polyethylene, polyurethane, poly(vinylchloride), and cyclic olefin polymer comprising at least 70% by weight of copolymer material composed of aliphatic cyclic or bicyclic hydrocarbon with 5 to 7 member ring or rings and ethylene or propylene, aliphatic cyclic or bicyclic hydrocarbon with a minimum of 5 member rings as the primary container wall.

In another illustrative embodiment, a material used to form the container is a polyolefin, a composition which is inexpensive and excellent in transparency and flexibility. Examples include polyethylene-base resins such as high-density polyethylene, medium-density polyethylene, high-pressure low-density polyethylene, low-density polyethylene, linear low-density polyethylene and ethylene-vinyl acetate copolymer; olefin-base elastomers such as ethylene-butadiene random copolymer; polypropylene-base resins such as polypropylene, ethylene-propylene random copolymer and α-olefin-propylene random copolymer; and mixtures thereof. Also, a vinyl chloride, an ethylene-vinyl acetate copolymer, a polyether sulfone, a cyclic polyolefin, a cyclic polyolefin copolymer, a styrene-base elastomer such as hydrogenated styrene ethylene butadiene copolymer, a mixture of two or more of these resins, or a mixture of such a resin with the above-described polyolefin-base resin can be used. These and a variety of other resins may be crosslinked for the purpose of elevating heat resistance, increasing barrier properties and the like.

Certain embodiments of the container materials (including the barrier coating layer discussed below) can include one or more biodegradable compositions, such as hydro-biodegradable and oxo-biodegradable compositions. A variety of polyester-based biodegradable systems have been characterized and studied. Polylactic acid (PLA), polyglycolic acid (PGA) and their copolymers polylactic-co-glycolic acid (PLGA) are some of the most well-characterized. See Uhrich, K. E.; Cannizzaro, S. M.; Langer, R. S, and Shakeshelf, K. M. “Polymeric Systems for Controlled Drug Release.” Chem. Rev. 1999, 99, 3181-3198 and Panyam J, Labhasetwar V. “Biodegradable nanoparticles for drug and gene delivery to cells and tissue.” Adv Drug Deliv Rev. 2003, 55, 329-47. Biodegradable systems based on polyorthoesters have also been investigated. See Heller, J.; Barr, J.; Ng, S. Y.; Abdellauoi, K. S, and Gurny, R. “Poly(ortho esters): synthesis, characterization, properties and uses.” Adv. Drug Del. Rev. 2002, 54, 1015-1039.

Certain embodiments of the invention are designed so that a user can visually inspect the medication to make sure, for example, that it is not crystallized or polymerized due to self association or denaturation, or that any other visually detectable change in the medication has occurred (e.g. oxidation of the active components). For example, in certain embodiments of the invention, the polymeric material and the coating layer are transparent. An illustrative polymeric material useful in this context comprises a copolymer material composed of aliphatic cyclic or bicyclic hydrocarbon with 5 to 7 member ring or rings and ethylene or propylene (e.g. as described in U.S. Pat. No. 6,680,091). The container is typically transparent so that it is possible to visually inspect the contents of the container to make sure that the medication has not crystallized or polymerized. While the walls of containers made from this material can provide a good permeation barrier against phenol, benzyl alcohol and m-cresol preservatives as well as water, this material does not in itself, however, provide a sufficient barrier against oxygen or other gasses.

A variety of containers commonly used to hold aqueous medications are encompassed by embodiments of the invention. In typical embodiments of the invention, the container comprises a medication storage vial, a medication reservoir disposed within a medical device, or infusion set tubing. In this context, the coating layer can be disposed, for example, on the outside of the container, on the inside of the container that contacts the fluid medication, or between a first layer and a second layer of polymeric material that forms the container. In addition, while medication storage vials and infusion set tubing that hold aqueous formulations are discussed as typical embodiments of the invention, other embodiments of the invention can be used to form coatings on a wide variety of different types of medical devices (e.g. implantable medical devices such as medication pumps, catheter shunts and the like) in order to function as barriers to a variety of aqueous fluids, for example, biological fluids that contact implantable medical devices (e.g. blood, sera, interstitial fluid and the like).

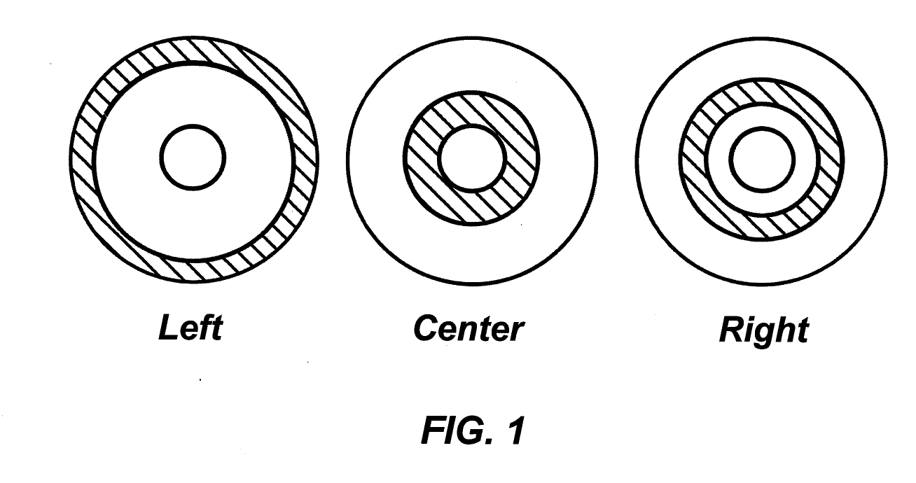

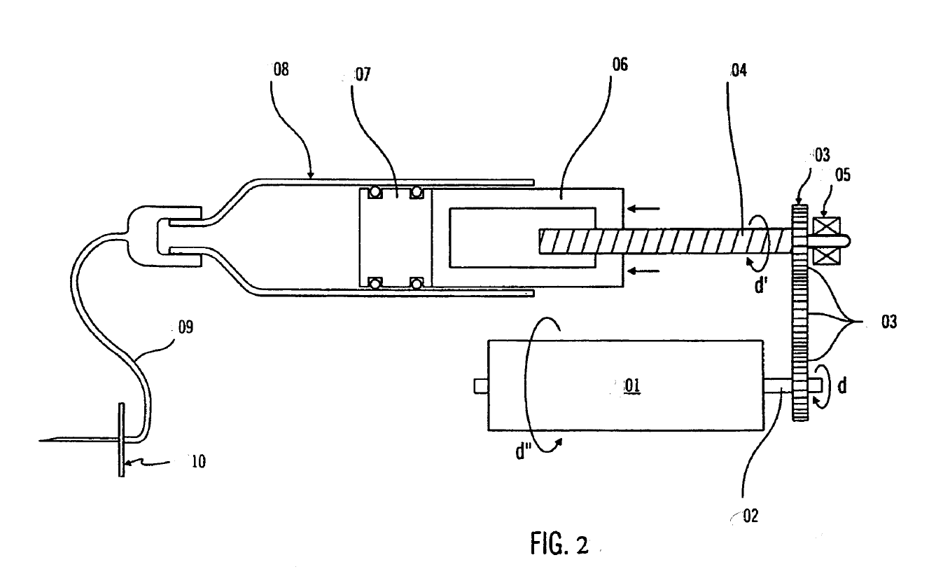

FIG. 1 provides a diagram of a coating layer (or layers) disposed, for example, on the outside of the container, on the inside of the container that contacts the fluid medication, or between a first layer and a second layer of polymeric material that forms the container. FIG. 2 provides a diagram of an illustrative medical device having containers (reservoir 08 and/or tubing 09) comprising embodiments disclosed herein. In FIG. 2, a motor 01 (or a motor with an attached gear box) has a drive shaft 02 engaged to drive a set of gears 03. The motor 01 generates a torque powering the drive shaft 02 in direction d. The drive shaft 02 rotates the gears 03 to transfer the torque to a lead screw 04, rotating the lead screw 04 in the direction d′. The lead screw 04 is mounted on a bearing 05 for support. The threads of the lead screw 04 are engaged with threads (not shown) in a slide 06. The slide 06 is engaged with a slot (not shown) in the housing (not shown) to prevent the slide 06 from rotating, but allowing it to translate along the length of the lead screw 04. Thus, the torque d′ of the lead screw 04 is transferred to the slide 06 causing the slide 06 to move in an axial direction, generally parallel to the drive shaft 02 of the motor 01. The slide 06 is in contact with a stopper 07 inside a reservoir 08. As the slide 06 advances, the stopper 07 is forced to travel in an axial direction inside the reservoir 08, forcing fluid from the reservoir 08, through tubing 09, and into an infusion set 10.

### Illustrative Coating Materials of the Invention

In embodiments of the invention, a surface of a container is coated with a layer of material selected for its ability to inhibit the diffusion of the compound and gases into or through the container. An illustrative embodiment of the invention is an aqueous container system comprising a container formed from a polymeric material, an aqueous solution disposed within the container and comprising a polypeptide combined with a compound selected for its ability to preserve a biological activity of the polypeptide. In this embodiment of the invention, a surface of the container is coated with a layer of material selected for its ability to inhibit binding of permeation info of diffusion of the compound and gases into or through the container. Closely related embodiments of the invention are designed to inhibit the ability of a compound within an aqueous formulation to bind (and/or permeate into) a surface of the container. A variety of polymeric compositions can be used to form the container, for example a polyethylene, a polypropylene, a polyester, a cyclic olefin, a polyurethane, a poly(vinylchloride), or a co-polymer material composed of aliphatic cyclic or bicylic hydrocarbons with 5 to 7 membered ring or rings, and ethylene or propylene. In addition, a variety of compositions can be used to form the coating layer or layers. Such compositions include, for example, one or more biodegradable compositions, such as hydro-biodegradable and oxo-biodegradable compositions. In illustrative embodiments of the invention, the coating layer comprises a poly(p-xylylene) compound, a silicon compound (e.g. silicone oxide), a fluoropolymer compound, a ethylene vinyl alcohol compound, a polyvinylidine compound, a tetrahedral amorphous carbon compound, or a metal compound (e.g. aluminum oxide) as well as combinations of two or more of these materials. Aspects of a number of these coating materials are discussed in the following paragraphs.

In some embodiments of the invention, the coating layer comprises a Parylene. Parylene is the tradename for a variety of poly(p-xylylene) polymers that can be used as moisture barriers, and electrical insulators due to properties such as their inertness and low coefficient of friction. Parylene exhibits a green chemistry, which is self-initiated (no initiator needed) and un-terminated (no termination group needed) with no solvent or catalyst required. There are a number of derivatives and isomers of parylene including: Parylene N (hydrocarbon), Parylene C (one chlorine group per repeat unit), Parylene D (two chlorine groups per repeat unit), Parylene AF-4 (generic name, aliphatic flourination 4 atoms), Parylene SF (Kisco product), Parylene HT (AF-4, SCS product), Parylene A (one amine per repeat unit, Kisco product), Parylene AM (one methylene amine group per repeat unit, Kisco product), Parylene VT-4 (generic name, fluorine atoms on the aromatic ring), Parylene CF (VT-4, Kisco product), and Parylene X (a cross-linkable version, not commercially available). Among them, Parylene C is the popular due to its combination of barrier properties, cost, and other manufacturing advantages. It is the most bio-accepted coating for stents, defibrillators, pacemakers and other devices permanently implanted into the body. Parylene N is a polymer manufactured from di-p-xylylene, a dimer synthesized from p-xylylene. Parylene N is an unsubstituted molecule. Heating [2.2]paracyclophane under low pressure (0.01-1 Torr) conditions gives rise to a diradical species which polymerizes when deposited on a surface. Until the monomer comes into contact with a surface it is in a gaseous phase and can access the entire exposed surface of a container.

In some embodiments of the invention, the coating layer comprises a silicon (e.g. a silicon oxide) or a silicone compound. Silicones (e.g. polymerized siloxanes or polysiloxanes) are mixed inorganic-organic polymers with the chemical formula [R2SiO]n, where R is organic groups such as methyl, ethyl, and phenyl. These materials consist of an inorganic silicon-oxygen backbone (—Si—O—Si—O—Si—O—) with organic side groups attached to the silicon atoms, which are four-coordinates. Although silicones contain silicon atoms, they are not made up exclusively of silicon, and have completely different physical characteristics from elemental silicon. By varying the —Si—O— chain lengths, side groups, and cross linking, silicones can be synthesized with a wide variety of properties and compositions. They can vary in consistency from liquid to gel to rubber to hard plastic. The most common siloxane is linear polydimethylsiloxane (PDMS). The second largest group of silicone materials is based on silicone resins, which are formed by branched and cage-like oligosiloxanes. As used herein, the term “silicone” therefore encompasses a large number of compounds based on polydialkylsiloxanes; the most common being trimethylsilyloxy terminated polydimethylsiloxanes.

In some embodiments of the invention, the coating material comprises a fluoropolymer compound. Fluoropolymers comprise a family of engineering plastics with multiple strong carbon-fluorine bonds, which are characterized by high thermal stability, almost universal chemical resistance and low friction. Fluoropolymers share the properties of fluorocarbons in that they are not as susceptible to the van der Waals force as hydrocarbons. This contributes to their non-stick and friction reducing properties. Also, they are stable due to the stability multiple carbon-fluorine bonds add to a chemical compound. Fluoropolymers may be mechanically characterized as thermosets or thermoplastics. Fluoropolymers can be homopolymers or copolymers. In some embodiments of the invention, the coating material comprises a polyvinylidine compound such as polyvinylidene fluoride. Polyvinylidene Fluoride, or PVDF is a highly non-reactive and pure thermoplastic fluoropolymer. PVDF is a specialty plastic material in the fluoropolymer family; it is used generally in applications requiring the highest purity, strength, and resistance to solvents, acids, bases and heat. In some embodiments of the invention, the coating material comprises an ethylene vinyl alcohol compound. Ethylene vinyl alcohol (EVOH) resin is a random copolymer of ethylene and vinyl alcohol. EVOH resins can be co-extruded with many types of polyolefins, polyamides, polystyrene and polyesters. In embodiments of the invention, container and/or coating compounds may be modified by one or more crosslinking compounds and/or processes. For example, compounds such as polyvinyl alcohol or copolymer of ethylene vinyl glycol can be cross-linked with one or more other compounds, for example a polyethyleneimine, a melamine derivatives, mixtures thereof and the like.

In some embodiments of the invention, the coating material comprises a tetrahedral amorphous carbon compound. Amorphous carbon is often abbreviated to aC for general amorphous carbon, a C:H for hydrogenated amorphous carbon, or to ta-C for tetrahedral amorphous carbon (also called diamond-like carbon). Diamond-like carbon (DLC) exists in a number of different forms of amorphous carbon materials that display some of the unique properties of diamond. They are usually applied as coatings to other materials that could benefit from some of those properties. By mixing these types in various ways, DLC coatings can be made that at the same time are amorphous, flexible, and yet purely sp3 bonded “diamond”. The hardest, strongest, and slickest is such a mixture, known as tetrahedral amorphous carbon, or ta-C. See, e.g. Finch et al., “Diamond like carbon, a barrier coating for polymers used in packaging applications”, Packaging Technology and Science Volume 9, pages 73-85, 1996 notes that this coating is flexible, very impermeable and adheres well to wide range of polymers. DLC therefore exhibits a number of desirable properties for a barrier coatings used in embodiments of the invention.

In some embodiments of the invention, the coating material comprises a metal compound. The terms “metal” or “metals” as used herein are meant to include one or more metals whether in the form of substantially pure metals, alloys or compounds such as oxides, oxynitrides, oxyborides, nitrides, borides, sulphides, halides or hydrides. Optionally, a metal used in the coating layer is one known to have an anti-microbial effect (e.g. Ag). Illustrative metals include Al, Ag, Au, Pt, and Pd. As discussed below, metal coatings are typically produced as thin films by vapor deposition techniques such as sputtering. Techniques to deposit metal-films are reviewed by R. F. Bunshah et al., “Deposition Technologies for Films and Coatings”, Noyes Publications, N.J., 1982 and by J. A. Thornton, “Influence of Apparatus Geometry and Deposition Conditions on the Structure and Topography of Thick Sputtered Coatings”, J. Vac. Sci. Technol., 11(4), 666-670, 1974.

### Illustrative Methods of the Invention

Embodiments of the invention include methods of inhibiting the loss of a biological activity of a polypeptide stored within a container by storing the polypeptide within an aqueous container system disclosed herein. An illustrative aqueous container system can comprise, for example, a container formed from a polymeric material, and an aqueous solution disposed within the container which includes a polypeptide combined with a compound selected for its ability to preserve a biological activity of the polypeptide. In such embodiments of the invention, a surface of the container is coated with a layer of material selected for its ability to inhibit diffusion of the compound and/or gases into or through the container so that the preservative activity and/or concentration of the compound is maintained in a manner that inhibits the loss of a biological activity of the polypeptide under a variety of conditions. Certain embodiments of the invention are used with polypeptides, that, at times during their use, are stored at temperatures above 5° C. or 10° C. (e.g. room temperature) for at least 24, 48, 72 or more hours prior to their administration to a patient. Some embodiments of the invention are used with polypeptides, that, at times during their use, are stored at room temperature for at least 28 days (room temperature is 59° to 86° F.).

In certain embodiments of the invention, the polypeptide comprises insulin, the preservative comprises a cresol and the container comprises a medication reservoir disposed within a medication infusion pump having dimensions smaller than 15×15 centimeters and designed to be worn by a user during daily activities (and/or infusion set tubing that is operatively coupled to such a reservoir). Optionally the medication infusion pump is suited for ambulatory use and is operably coupled to an interface that facilitates its attachment to the user, wherein the interface comprises a clip, a strap, a snap, a clamp or an adhesive strip.

Embodiments of the invention also include methods of making an aqueous container system. One such embodiment comprises, for example, the steps of forming a container from a polymeric material, coating a surface of the container with a layer of material selected for its ability to function as a barrier that inhibits diffusion of compounds and gases into or through the container such that compounds can maintained at a concentration sufficient to inhibit loss of a biological activity of a polypeptide disposed within the container. A solution is then placed within the container, with illustrative solutions comprising water, and a polypeptide combined with a compound selected for its ability to preserve a biological activity of the polypeptide.

In illustrative methods for making an aqueous container system the surface of the container can be modified by applying a thin film of material. In this context, a variety of well know processes are used to apply a thin coating of materials. Such thin film formation methods include, for example, spin coating, which typically comprises adding a coating solution to a substrate to be coated and drawing the coating solution thereon by centrifugal force, so as to form a thin film. Thin film methods can also comprise, for example, a process including plasma surface treatment. Optionally, a surface of the container is modified by a process comprising chemical and/or physical vapor deposition. In certain embodiments of the invention, the surface of the container is coated with the layer of material under a vacuum, for example at a pressure at least 10% below atmospheric pressure.

In one illustrative embodiments of the invention, the surface of a container is modified by a process comprising plasma surface modification. In a specific illustrative embodiment of the invention, infusion set tubing is plasma treated to modify its constituent polymers such as a fluoropolymer, and/or to apply a layer of material such as aluminum oxide (AlOx) or silicon oxide (SiOx) in a manner that facilities the generation of a barrier layer that functions to inhibit the permeability of the tubing to gases and/or preservatives (see, e.g. U.S. Pat. Nos. 6,824,872; 6,265,038 and 6,635,571, the contents of which are incorporated herein by reference).

While embodiments of the invention can use atmospheric plasma surface modification, plasma processing of polymers is normally carried out at sub-atmospheric pressures, which allows processing to be conducted at lower temperatures than atmospheric-pressure plasmas (e.g., near room temperature, in some cases). Plasma processes useful with embodiments of the invention include radiofrequency plasmas (e.g., capacitively coupled plasmas, inductively coupled plasmas, helicon plasmas, etc.) and microwave plasmas (e.g., electron cyclotron resonance plasmas, etc.), among others. Various energetic species are associated with plasmas, including ions, electrons and photons (including UV photons). Where the magnitude of the energy transfer from the plasma is higher than the binding energy of certain orbital electrons in the polymer, the polymer will be ionized, leading to molecular fragmentation into small fragments that contain free radicals. Where the magnitude of the energy transfer from the plasma is lower than the binding energy, on the other hand, certain electrons in the polymer are raised to an excited upper orbital, followed by dissociation, producing radicals at the polymer surface (see, e.g., N. Inagaki, Ph.D., Plasma Surface Modification and Plasma Polymerization). Illustrative processes comprising plasma surface modification are described in N. Inagaki, Plasma Surface Modification and Plasma Polymerization, Technomic Publishing Company, Inc. 1996; and L. Hanley et al., “The growth and modification of materials via ion-surface processing”, Surface Science 500, 2002.

In some embodiments of the invention, the surface of a container is modified by a process comprising vapor deposition techniques such as sputtering. Such process can include physical vapor deposition and chemical vapor deposition. Physical vapor deposition (PVD) is a variety of vacuum deposition and is a general term used to describe any of a variety of methods to deposit thin films by the condensation of a vaporized form of the material onto various surfaces (e.g., onto semiconductor wafers). The coating method involves purely physical processes such as high temperature vacuum evaporation or plasma sputter bombardment rather than involving a chemical reaction at the surface to be coated as in chemical vapor deposition. Chemical vapor deposition (CVD) is a chemical process used to produce high-purity, high-performance solid materials. In a typical CVD process, the substrate is exposed to one or more volatile precursors, which react and/or decompose on the substrate surface to produce the desired deposit. Illustrative processes comprising vapor deposition are described in Smith, Donald (1995): Thin-Film Deposition: Principles and Practice. MacGraw-Hill; and Bunshah, Roitan F. (editor). Handbook of Deposition Technologies for Films and Coatings: Science, Technology and Applications, second edition.

Materials science and process technology series. Park Ridge, N.J.: Noyes Publications, 1994. Many changes and modifications within the scope of the present invention may be made without departing from the spirit thereof, and the invention includes all such modifications. For example it will be readily apparent to those of skill in this art that a wide variety of aqueous container systems can be made by selecting and combining the various different elements of these systems that are disclosed herein.

1. An aqueous container system comprising:
a container formed from a polymeric material;
an aqueous solution disposed within the container and comprising a polypeptide combined with a compound selected for its ability to preserve a biological activity of the polypeptide;
wherein a surface of the container is coated with a layer of material selected for its ability inhibit diffusion or permeation of the compound and gases into or through the container.

2. The aqueous container system of claim 1, wherein the polymeric material comprises:
a polyethylene; a polypropylene; a polyester; a cyclic olefin; a polyurethane; a poly(vinylchloride); a polyamide; a polycarbonate; a polyacrylate; a polystyrene; a polyimide; a polysulfone; a poly ether ether ketone (PEEK); a silicone; or
a co-polymer material composed of aliphatic cyclic or bicylic hydrocarbons with 5 to 7 membered ring or rings, and ethylene or propylene.

3. The aqueous container system of claim 1, wherein the coating layer comprises:
a poly(p-xylylene) compound;
a silicon compound;
a fluoropolymer compound;
a ethylene vinyl alcohol compound;
a polyvinylidine compound;
a tetrahedral amorphous carbon compound;
or a metal compound.

4. The aqueous container system of claim 1, wherein the compound comprises an antimicrobial agent.

5. The aqueous container system of claim 1, wherein the compound comprises a phenol, a benzyl alcohol or a cresol.

6. The aqueous container system of claim 1, wherein the polypeptide comprises an insulin.

7. The aqueous container system of claim 1, wherein the container comprises a medication reservoir or infusion set tubing.

8. The aqueous container system of claim 1, wherein the coating layer is disposed between a first layer and a second layer of polymeric material.

9. The aqueous container system of claim 1, wherein the polymeric material and the coating layer are transparent.

10. A method of inhibiting loss of a biological activity of a polypeptide stored within a container, comprising storing the polypeptide within an aqueous container system comprising:
a container formed from a polymeric material;
an aqueous solution disposed within the container and comprising the polypeptide combined with a compound selected for its ability to preserve a biological activity of the polypeptide;
wherein a surface of the container is coated with a layer of material selected for its ability to inhibit diffusion of the compound and gases into or through the container so that the compound is maintained at a concentration sufficient to inhibit loss of a biological activity of the polypeptide.

11. The method of claim 10, wherein the polypeptide is stored at room temperature for at least 24, 28 or 72 hours prior to administration.

12. The method of claim 10, wherein the container is a medication reservoir disposed within a medication infusion pump having dimensions smaller than 15×15 centimeters and designed to be worn by a user during daily activities.

13. The method of claim 12, wherein, wherein the medication infusion pump is operably coupled to an interface that facilitates its attachment to the user, wherein the interface comprises a clip, a strap, a snap, a clamp or an adhesive strip.

14. The method of claim 10, wherein the polypeptide comprises insulin and the container comprises a medication reservoir or infusion set tubing.

15. A method of making an aqueous container system comprising:
forming a container from a polymeric material;
coating a surface of the container with a layer of material selected for its ability to function as a barrier that inhibits diffusion of compounds and gases into or through the container such that compounds can maintained at a concentration sufficient to inhibit loss of a biological activity of a polypeptide disposed within the container;
disposing an aqueous solution within the container, wherein the aqueous solution comprises a polypeptide combined with a compound selected for its ability to preserve a biological activity of the polypeptide;
so that an aqueous container system is made.

16. The method of claim 15, wherein the surface of the container is modified a process comprising plasma surface treatment.

17. The method of claim 15, wherein the surface of the container is modified by a process comprising vapor deposition.

18. The method of claim 15, wherein the surface of the container is coated with the layer of material at a pressure at least 10% below atmospheric pressure.

19. The method of claim 15, wherein the surface of the container is coated with the layer of material comprising:
a poly(p-xylylene) compound;
a silicon compound;
a fluoropolymer compound;
a ethylene vinyl alcohol compound;
a polyvinylidine compound;
a tetrahedral amorphous carbon compound;
or a metal compound; and
the polymeric container material comprises:
a polyethylene;
a polypropylene;
a polyester;
a cyclic olefin;
a polyurethane;
a poly(vinylchloride); or
a co-polymer material composed of aliphatic cyclic or bicylic hydrocarbons with 5 to 7 membered ring or rings, and ethylene or propylene.

20. The method of claim 15, wherein the polypeptide comprises an insulin and the container comprises a medication reservoir or infusion set tubing.
